(19) United States
(12) Patent Application Publication
(10) Pub. No.: US 2021/0151128 A1
(43) Pub. Date: May 20, 2021

(54) Learning Method, Mixing Ratio Prediction Method, and Prediction Device

(71) Applicant: Preferred Networks, Inc., Tokyo (JP)

(72) Inventors: Motoki Abe, Tokyo (JP); Daisuke Okanohara, Tokyo (JP); Kenta Oono, Tokyo (JP); Mizuki Takemoto, Tokyo (JP)

(73) Assignee: Preferred Networks, Inc., Tokyo (JP)

(21) Appl. No.: 17/134,802

(22) Filed: Dec. 28, 2020

Related U.S. Application Data
(63) Continuation of application No. PCT/JP2019/025676, filed on Jun. 27, 2019.

(30) Foreign Application Priority Data
Jun. 29, 2018 (JP) 2018-124385

(51) Int. Cl. G16B 40/00

(52) U.S. Cl. CPC G16B 40/00

(57) ABSTRACT

A learning method of a mixing ratio prediction of element comprising causing a machine learning model to learn to output, in response to input of group expression level data indicating an expression level of each element in a group to be predicted, a mixing ratio of an element contained in the group, wherein in the causing a machine learning model to learn, a virtual mixing ratio that differs among a plurality of pieces of learning data is set as desired, and a learning dataset is used, the learning dataset including data generated, for each piece of the learning data, by obtaining a virtual expression level that is a virtual expression level corresponding to the virtual mixing ratio based on original data indicating an expression level in each element.

### CROSS-REFERENCE TO RELATED APPLICATIONS

This application is a continuation of International Application No. PCT/JP2019/025676, with an international filing date of Jun. 27, 2019, which claims priority to Japanese Patent Application No. 2018-124385 filed on Jun. 29, 2018, each of which is incorporated herein by reference in its entirety.

### TECHNICAL FIELD

The present disclosure relates to a learning method, a mixing ratio prediction method, and a learning device.

### BACKGROUND

In the development of, for example, immunotherapy, it is important to understand changes in immune state due to a disease. Under these circumstances, in recent years, a method for predicting a mixing ratio of each cell type (type of cell) in tissue has been studied using data indicating an expression level (gene expression level) of each gene in an immune cell. In such a study, a cell group containing a plurality of types of cells (hereinafter, referred to as a “bulk cell”) is used for prediction of a mixing ratio of each cell type contained in the bulk cell, for example.

### SUMMARY

In order to achieve the above-described object, an embodiment of the present invention includes causing a machine learning model to learn to output, in response to input of cell group expression level data indicating an expression level of each gene in a cell group to be predicted, a mixing ratio of a cell contained in the cell group. In the causing a machine learning model to learn, a virtual mixing ratio that differs among a plurality of pieces of learning data is set as desired, and a learning dataset is used, the learning dataset including data generated, for each piece of the learning data, by obtaining a virtual expression level that is a virtual gene expression level corresponding to the virtual mixing ratio based on original data indicating a gene expression level in each cell type.

### BRIEF DESCRIPTION OF DRAWINGS

FIG. 1 is a diagram for describing a concept of how a mixing ratio prediction device according to an embodiment of the present invention makes predictions.

FIG. 2 is a diagram for describing learning data used in the mixing ratio prediction device according to the embodiment of the present invention.

FIG. 3 is a diagram showing how to generate the learning data for the mixing ratio prediction device according to the embodiment of the present invention.

FIG. 4 is a diagram showing an example of a function configuration of the mixing ratio prediction device according to the embodiment of the present invention.

FIG. 5 is a diagram showing an example of a hardware configuration of the mixing ratio prediction device according to the embodiment of the present invention.

FIG. 6 is a flowchart showing an example of a learning dataset creation process.

FIG. 7 is a flowchart showing an example of a learning process.

FIG. 8 is a flowchart showing an example of a prediction process.

FIG. 9A is a diagram showing examples of comparison with a method in the related art.

FIG. 9B is a diagram showing examples of comparison with a method in the related art.

### DETAILED DESCRIPTION

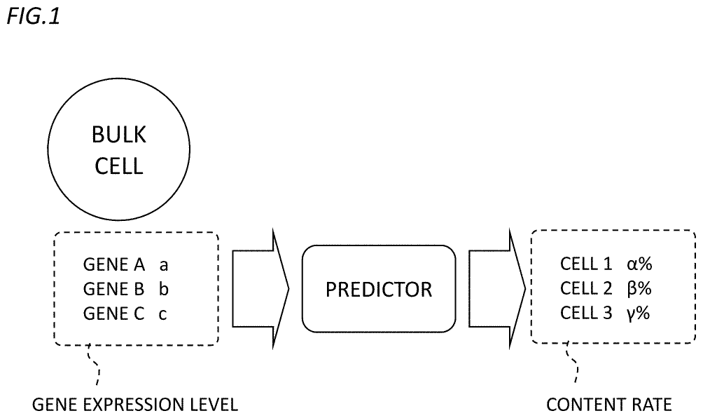

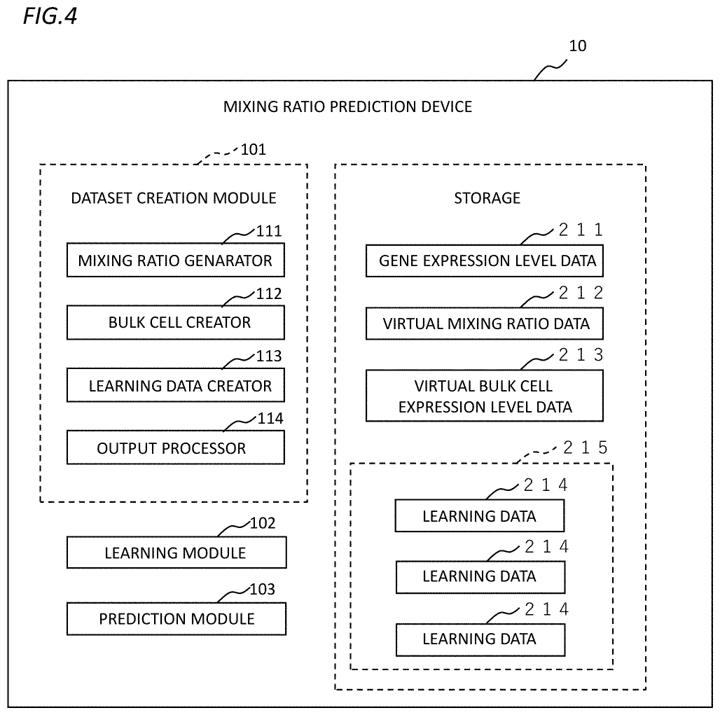

An embodiment of the present invention will be described in detail below with reference to the drawings. According to the embodiment of the present invention, a mixing ratio prediction device 10 capable of predicting a mixing ratio of each cell type contained in a bulk cell with high accuracy will be described. First, a concept of how the mixing ratio is predicted will be described with reference to FIGS. 1 to 3, and then a configuration of the mixing ratio prediction device 10 will be described in detail with reference to FIG. 4. Herein, the mixing ratio refers to a proportion of each cell type contained in the bulk cell. Further, the bulk cell refers to a cell group containing a plurality of types of cells. The mixing ratio may be referred to as, for example, a content rate or an abundance ratio.

Note that, as an example according to the embodiment of the present invention, a sample cell containing a plurality of types of immune cells is used as the bulk cell. Note that the bulk cell may contain various types of cells (for example, cancer cells, muscle cells, nerve cells, etc.) other than such immune cells.

As shown in FIG. 1, the mixing ratio prediction device 10 according to the embodiment of the present invention is configured to input data indicating gene expression levels in the bulk cell (hereinafter, also referred to as “bulk cell expression level data”) to a predictor implemented by, for example, a learned neural network to output data indicating the mixing ratio of each cell type contained in the bulk cell (hereinafter, also referred to as “mixing ratio prediction data”).

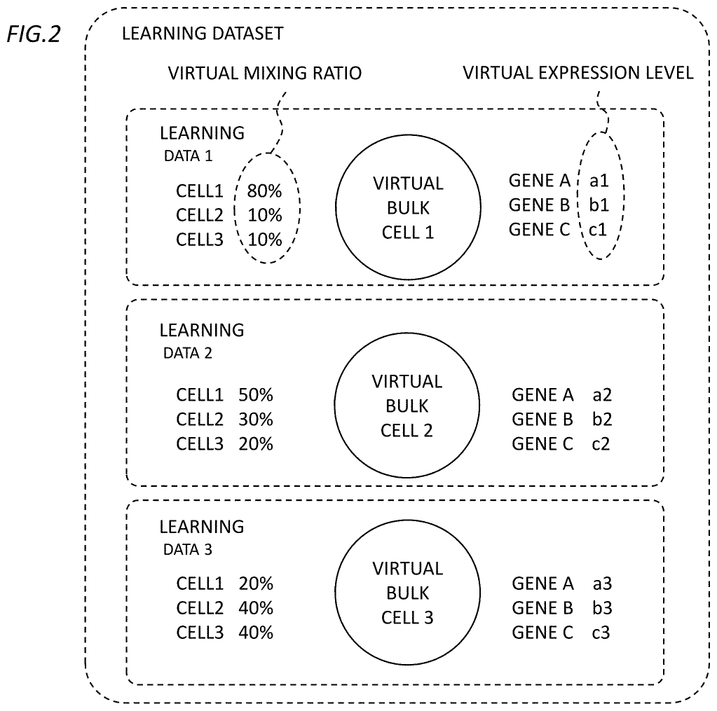

As shown in FIG. 2, the mixing ratio prediction device 10 causes a machine learning model to learn based on a learning dataset including a plurality of pieces of learning data each having a “virtual mixing ratio” and a “virtual expression level”. As shown in FIG. 2, each piece of learning data is virtual data generated for a corresponding virtual bulk. In the example shown in FIG. 2, the learning dataset includes learning data 1 to 3, but no limitation is imposed on the number of pieces of learning data included in the learning dataset.

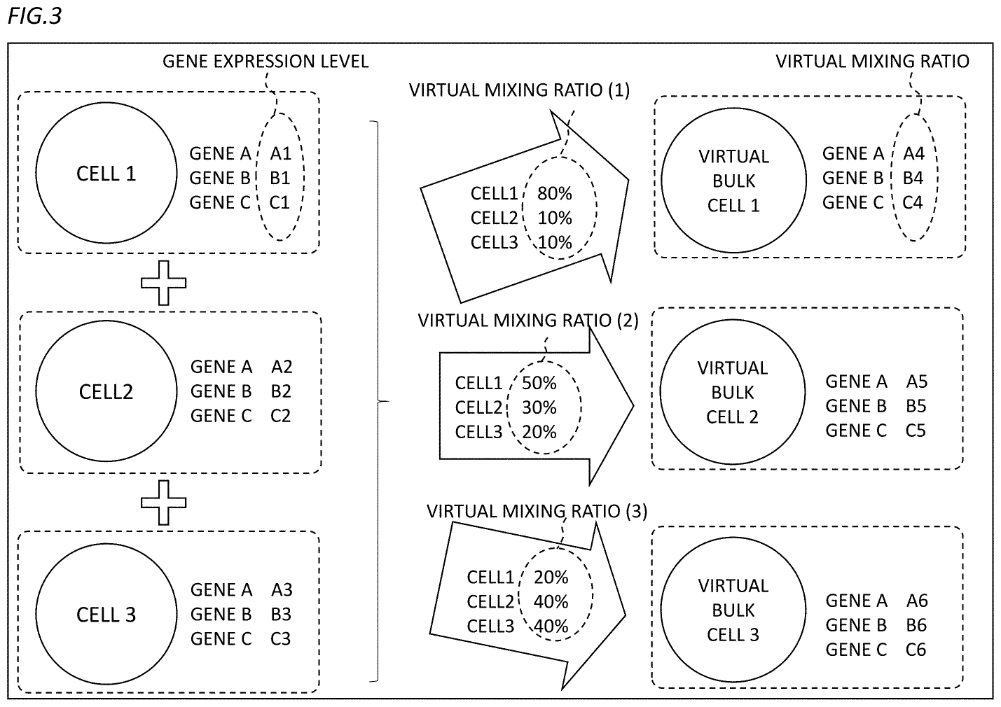

FIG. 3 shows a concept of how the learning data is generated in the mixing ratio prediction device 10. The mixing ratio prediction device 10 first generates, in order to predict the mixing ratio of each cell type contained in the bulk cell, a virtual bulk cell that is a bulk cell virtually generated based on gene expression levels in a plurality of cells. Specifically, FIG. 3 shows an example where “virtual bulk cell 1”, “virtual bulk cell 2”, and “virtual bulk cell 3” are generated from “cell 1”, “cell 2”, and “cell 3”. Herein, the “virtual bulk cell” does not actually exist, but is virtually obtained through calculation for generating the learning data used for prediction of the mixing ratio to be described later.

In the example shown in FIG. 3, each cell is made up of “gene A”, “gene B”, and “gene C”. Specifically, in “cell 1”, it is assumed that the gene expression level of the gene A is denoted by “A1”, the gene expression level of the gene B is denoted by “B1”, and the gene expression level of the gene C is denoted by “C1”. Further, in “cell 2”, it is assumed that the gene expression level of the gene A is denoted by “A2”, the gene expression level of the gene B is denoted by “B2”, and the gene expression level of the gene C is denoted by “C2”. Furthermore, in “cell 3”, it is assumed that the gene expression level of the gene A is denoted by “A3”, the gene expression level of the gene B is denoted by “B3”, and the gene expression level of the gene C is denoted by “C3”. Note that the cells 1 to 3 and the genes A to C are names abbreviated for explanation. Further, the number and types of genes that make up an actual cell also differ.

First, the mixing ratio prediction device 10 sets a virtual mixing ratio of each cell. In the example shown in FIG. 3, as the virtual mixing ratio, (1) “cell 1:80%, cell 2:10%, cell 3:10%”, (2) “cell 1:50%, cell 2:30%, cell 3:20%”, and (3) “cell 1:20%, cell 2:40%, cell 3:40%” are set.

Subsequently, the mixing ratio prediction device 10 mixes “cell 1” at 80%, “cell 2” at 10%, and “cell 3” at 10% in accordance with the virtual mixing ratio (1) to generate “virtual bulk cell 1”. Then, the mixing ratio prediction device 10 uses the respective proportions A1 to C1 of the genes A to C making up the cells 1 to 3 to determine virtual expression levels A4 to C4 representing the respective virtual expression levels of the genes A to C making up “virtual bulk cell 1”.

Similarly, the mixing ratio prediction device 10 generates “virtual bulk cell 2” at the virtual mixing ratio (2) and determines respective virtual expression levels A5 to C5 of the genes A to C. Further, the mixing ratio prediction device 10 generates “virtual bulk cell 3” at the virtual mixing ratio (3) and determines respective virtual expression levels A6 to C6 of the genes A to C.

This allows the mixing ratio prediction device 10 according to the present invention to use the virtual mixing ratio and the virtual expression level as the learning data even when a sufficient volume of bulk cell information cannot be obtained as the learning data and to predict the cell mixing ratio from the gene expression levels in the bulk cell. That is, the mixing ratio prediction device 10 can make the prediction with the learning data that is virtual information obtained through the generation process, instead of data obtained through measurement or the like. In other words, the mixing ratio prediction device 10 uses a new method in which learning is made based on virtual data, instead of learning processes in the related art.

A description will be given below of “learning dataset creation process” of creating a dataset (learning dataset) for use in learning a predictor, “learning process” of causing the predictor to learn using the learning dataset, and “prediction process” of predicting, by the predictor, the mixing ratio of each cell type contained in the bulk cell.

Note that, as an example according to the embodiment of the present invention, a case where the predictor is implemented by a learned neural network will be described. Note that the predictor may be implemented by not only such a learned neural network, but also various machine learning models such as a decision tree and a support vector machine.

### Function Configuration

Next, a description will be given of a function configuration of the mixing ratio prediction device 10 according to the embodiment of the present invention with reference to FIG. 4. FIG. 4 is a diagram showing an example of the function configuration of the mixing ratio prediction device 10 according to the embodiment of the present invention.

As shown in FIG. 4, the mixing ratio prediction device 10 according to the embodiment of the present invention includes a dataset creation module 101, a learning module 102, and a prediction module 103. Further, the mixing rate prediction device 10 is capable of storing and using, in a storage device, various pieces of data such as gene expression level data 211, virtual mixing ratio data 212, virtual expression level data (hereinafter, also referred to as “virtual bulk cell expression level data”) 213, and learning data 214. The storage device shown in FIG. 4 is a storage means including a RAM 205, a ROM 206, a secondary storage device 208, and the like, and each piece of data can be stored in any of the storage means.

The dataset creation module 101 executes the learning dataset creation process. That is, the dataset creation module 101 uses, as input, the gene expression level data 211 of each cell type to create a learning dataset 215. Herein, the dataset creation module 101 includes a mixing ratio generator 111, a bulk cell creator 112, and a learning data creator 113.

The mixing ratio generator 111 generates the virtual mixing ratio data 212 indicating the virtual mixing ratio of each cell type contained in the bulk cell. At this time, the mixing ratio generator 111 generates a plurality of pieces of virtual mixing ratio data 212.

The bulk cell creator 112 creates, for each piece of virtual mixing ratio data 212, the virtual bulk cell expression level data 213 indicating the gene expression levels in the virtual bulk cell from the gene expression level data 211 of each cell type and the virtual mixing ratio data 212.

The learning data creator 113 creates, for each piece of virtual mixing ratio data 212, a set of the virtual bulk cell expression level data 213 and the virtual mixing ratio data 212 as the learning data 214. As a result, the learning dataset 215 made up of a plurality of pieces of learning data 214 is created. Note that, in the example shown in FIG. 4, the learning dataset 215 is made up of three pieces of learning data 214, but as described above, no limitation is imposed on the number of pieces of learning data 214 included in the learning dataset 215.

The learning module 102 executes the learning process. That is, the learning module 102 updates parameters of the neural network based on each piece of learning data 214 included in the learning dataset 215. This causes the neural network to learn to implement the predictor.

The prediction module 103 is a predictor implemented by the learned neural network and executes the prediction process. That is, the prediction module 103 outputs, upon receipt of bulk cell expression level data indicating the gene expression levels in the bulk cell as input, mixing ratio prediction data indicating a predicted value of the mixing ratio of each cell type contained in the bulk cell.

Note that, in the example shown in FIG. 4, a case where one mixing ratio prediction device 10 includes three function modules, the dataset creation module 101, the learning module 102, and the prediction module 103, has been given, but a plurality of devices may include the function modules in a distributed manner. For example, the mixing ratio prediction device 10 according to the embodiment of the present invention may be made up of a dataset creation device including the dataset creation module 101 and a prediction device including the learning module 102 and the prediction module 103. Further, the prediction device may be made up of a device that executes only the learning process and a device that executes only the prediction process. cl Hardware Configuration

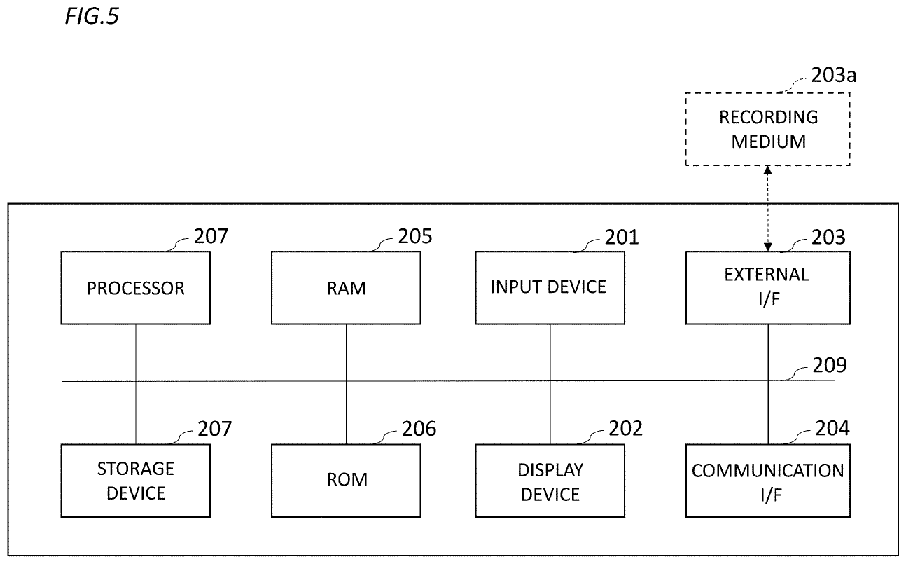

Next, a description will be given of a hardware configuration of the mixing ratio prediction device 10 according to the embodiment of the present invention with reference to FIG. 5. FIG. 5 is a diagram showing an example of the hardware configuration of the mixing ratio prediction device 10 according to the embodiment of the present invention.

As shown in FIG. 5, the mixing ratio prediction device 10 according to the embodiment of the present invention includes an input device 201, a display device 202, an external I/F 203, a communication I/F 204, and the random access memory (RAM) 205, the read only memory (ROM) 206, a processor 207, and the secondary storage device 208. Such hardware components are interconnected on a bus 209.

The input device 201 is, for example, a keyboard, a mouse, or a touch screen and is used by a user to input various operations. The display device 202 is, for example, a display and displays various process results from the mixing ratio prediction device 10. Note that the mixing ratio prediction device 10 need not include at least either the input device 201 or the display device 202.

The external I/F 203 is an interface with an external device. Examples of the external device include a recording medium 203a and the like. The mixing ratio prediction device 10 is capable of reading from or writing to the recording medium 203a and the like via the external I/F 203. The recording medium 203a may record at least one program and the like by which each function module (that is, the dataset creation module 101, the learning module 102, and the prediction module 103) of the mixing ratio prediction device 10 is implemented.

Examples of the recording medium 203a include a flexible disk, a compact disc (CD), a digital versatile disk (DVD), a secure digital (SD) memory card, and a universal serial bus (USB) memory card.

The communication I/F 204 is an interface for connecting the mixing ratio prediction device 10 to a communication network. At least one program by which each function module of the mixing ratio prediction device 10 is implemented may be acquired (downloaded) from a predetermined server device or the like via the communication I/F 204.

The RAM 205 is a volatile semiconductor memory that temporarily retains the program and data. The ROM 206 is a non-volatile semiconductor memory capable of retaining the program and data even when power is removed. The ROM 206 stores, for example, settings on an operating system (OS) and settings on the communication network.

The processor 207 is a processor such as a central processing unit (CPU) or a graphics processing unit (GPU) that loads a program and data from the ROM 206, the secondary storage device 208, or the like onto the RAM 205 and executes a corresponding process. Each function module of the mixing ratio prediction device 10 is implemented, for example, by the processor 207 executing at least one program stored in the secondary storage device 208. The mixing ratio prediction device 10 may include both the CPU and the GPU as the processor 207, or alternatively, may include only either the CPU or the GPU.

The secondary storage device 208 is a non-volatile storage device such as a hard disk drive (HDD) or a solid state drive (SSD) that stores the program and data. In the secondary storage device 208, for example, the OS, various application software, at least one program by which each function module of the mixing ratio prediction device 10 is implemented, and the like are stored.

The mixing ratio prediction device 10 according to the embodiment of the present invention that has the hardware configuration shown in FIG. 5 is capable of executing various processes to be described later. Note that, with reference to the example shown in FIG. 5, the configuration where the mixing ratio prediction device 10 according to the embodiment of the present invention is implemented by a single device (computer) has been described, but the present invention is not limited to such a configuration. The mixing ratio prediction device 10 according to the embodiment of the present invention may be implemented by a plurality of devices (computers).

### Learning Dataset Creation Process

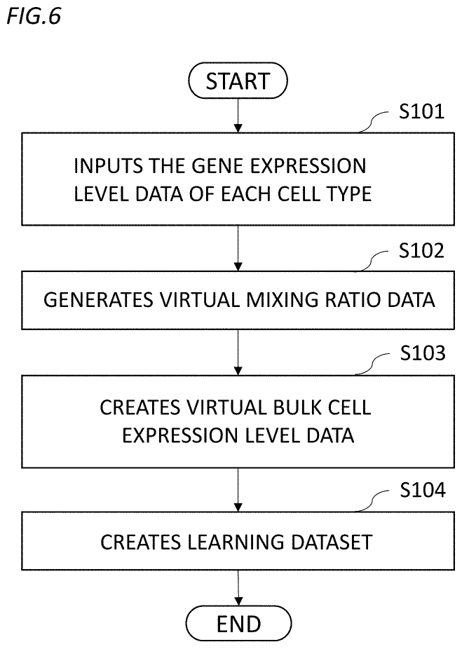

Next, a description will be given of the learning dataset creation process with reference to FIG. 6. FIG. 6 is a flowchart showing an example of the learning dataset creation process.

First, the dataset creation module 101 acquires the gene expression level data of each cell type (step S101). Herein, when the total number of gene types is denoted by M, and the total number of cell types is denoted by N, gene expression level data xₙ of a cell type n (1≤n≤N) is represented by an M-dimensional vector. That is, with the expression level of a gene M (1≤m≤M) in the cell type n denoted by xₘₙ, the gene expression level data xₙ is represented as xₙ=(x₁ₙ, . . . , x_{Mn})^{t}. Note that t denotes transpose.

As such gene expression level data of each cell type, for example, LM22 dataset may be used. The LM22 dataset is a set of data that results from measuring the expression levels of 547 types of genes in each of 22 types of homogeneous immune cells. For details of the LM22 dataset, refer to, for example, “Robust enumeration of cell subsets from tissue expression profiles”, Aaron M. Newman et al., Nature Methods 2015 May; 12(5): 453-457. In addition to the LM22 dataset, the gene expression level data of each cell type can also be obtained through, for example, single-cell RNA-Seq analysis.

The following description will be given on the assumption that gene expression level data x₁, . . . , x_{N} in which expression levels of M types of genes in N cell types are represented by an M-dimensional vector has been input.

The mixing ratio generator 111 of the dataset creation module 101 generates a plurality of pieces of virtual mixing ratio data (step S102). Herein, when the number of pieces of generated virtual mixing ratio data is denoted by P, the p(1≤p≤P)-th virtual mixing ratio data aₚ is represented by an N-dimensional vector (that is, a vector having dimensions as many as the total number of cell types). That is, with a mixing ratio of the cell type n (1≤n≤N) contained in the bulk cell denoted by aₙₚ, the virtual mixing ratio data aₚ is represented as aₚ=(a₁ₚ, . . . , a_{Np})^{t}. Therefore, the mixing ratio generator 111 generates, for each p, random numbers a₁ₚ, . . . , a_{Np} that satisfy a₁ₚ+ . . . +a_{Np}=1 and that each fall within a range of 0 to 1 to generate P pieces of virtual mixing ratio data a₁, . . . , aₚ. Note that P may be any natural number determined by the user.

Next, the bulk cell creator 112 of the dataset creation module 101 creates, for each piece of virtual mixing ratio data, virtual bulk cell expression level data from the gene expression level data of each cell type and the virtual mixing ratio data (step S103). Herein, the bulk cell creator 112 performs, with the gene expression level data x₁, . . . , x_{N} of each cell type represented as a matrix X=(x₁, . . . , x_{N}) that is a column vector, for example, a matrix product with the matrix X and the virtual mixing ratio data aₚ to create the virtual bulk cell expression level data yₚ. That is, the bulk cell creator 112 calculates yₚ=Xaₚ for p=1, . . . , P. As a result, M-dimensional vectors y₁, . . . , yₚ are obtained. Each yₚ represents the expression levels of M types of genes in the virtual bulk cell p.

Note that the bulk cell creator 112 may calculate yₚ=Xbₚ using virtual mixing ratio data bₚ that results from normalizing values obtained by multiplying the virtual mixing ratio data aₚ by predetermined noise to create the virtual bulk cell expression level data yₚ. The virtual mixing ratio data bₚ is created by, for example, multiplying each element aₙₚ (1≤n≤N) of aₚ by the predetermined noise (for example, salt pepper noise, lognormal noise, etc.) and then performing normalization such that the sum of the elements aₙₚ (1≤n≤N) multiplied by the noise is equal to 1.

Note that when the virtual bulk cell expression level data yₚ=Xbₚ based on the virtual mixing ratio data bₚ described above is created, the learning data creator 113 sets, for p=1, . . . , P, a set (yₚ, aₚ) of the virtual bulk cell expression level data yₚ=Xbₚ and the virtual mixing ratio data aₚ before being multiplied by the noise as learning data.

As described above, in the mixing ratio prediction device 10 according to the embodiment of the present invention, a learning dataset D={(yₚ, aₚ)|p=1, . . . , P} is created from the gene expression level data (for example, LM22 dataset, etc.) of each cell type obtained through actual measurement. Herein, as described above, yₚ denotes data indicating the gene expression levels in the virtual bulk cell, and aₚ denotes data indicating the mixing ratio of each cell type contained in the virtual bulk cell (that is, target variable data). As will be described later, this learning dataset D is used to cause the neural network to learn to implement the predictor.

Note that, in step S101 described above, a plurality of pieces of gene expression level data of the same cell type may be input. For example, gene expression level data x₁ and x₁′ of a cell type i may be input. In this case, it may be required that the above-described steps S103 and S104 be executed on gene expression level data x₁, . . . , xᵢ, . . . , x_{N} and gene expression level data x₁, . . . , xᵢ′, . . . , x_{N}. As a result, learning datasets D={(yₚ, aₚ)|p=1, . . . , P} and D′={(yₚ′, aₚ)|p=1, . . . , P} are created. Therefore, in this case, these learning datasets D and D′ may be used to cause the neural network to learn to implement the predictor. The same applies to a case where three or more pieces of gene expression level data of the same cell type are input.

### Learning Process

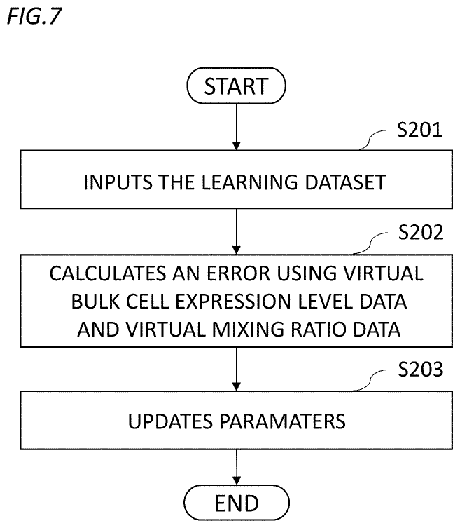

Next, a description will be given of a learning process with reference to FIG. 7. FIG. 7 is a flowchart showing an example of the learning process. Note that when a plurality of learning datasets are created in the above-described learning dataset creation process, it may be required that the following steps S201 to S203 be executed on each learning dataset, for example.

First, the learning module 102 inputs the learning dataset D={(yₚ, aₚ)|p=1, . . . , P} (step S201).

Next, the learning module 102 calculates an error using a predetermined error function by using each piece of learning data (yₚ, aₚ) contained in the learning dataset D (step S202). That is, the learning module 102 inputs the virtual bulk cell expression level data yₚ into the prediction module 103 (that is, an unlearned neural network) and obtains output data aₚ\hat{ } indicating the mixing ratio of each cell type contained in the virtual bulk cell p. Then, the learning module 102 calculates an error between the output data aₚ\hat{ } and the target variable data aₚ using the predetermined error function. Herein, as the error function, for example, softmax cross entropy, mean squared error, or the like is used.

Next, the learning module 102 updates the parameters of the neural network based on the error calculated in step S202 described above (step S203). That is, the learning module 102 updates the parameters by using, for example, backpropagation or the like to minimize the error. This causes the neural network to learn to implement the predictor.

As described above, the mixing ratio prediction device 10 according to the embodiment of the present invention is capable of acquiring the learned neural network by which the predictor is implemented.

### Prediction Process

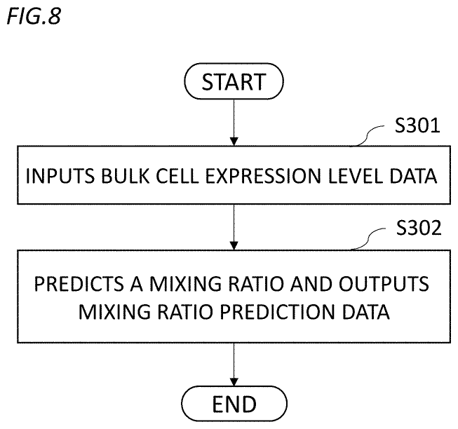

Next, a description will be given of a prediction process with reference to FIG. 8. FIG. 8 is a flowchart showing an example of prediction process.

The prediction module 103 inputs bulk cell expression level data y (step S301). Note that the bulk cell expression level data y can be obtained, for example, through measurement of gene expression levels in the bulk cell by a known method (for example, analysis using DNA microarray, RNA-Seq analysis, etc.).

Next, the prediction module 103 causes the predictor to predict a mixing ratio of each cell type contained in the bulk cell corresponding to the bulk cell expression level data y and outputs mixing ratio prediction data a indicating the predicted mixing ratios (step S302). As a result, the mixing ratio prediction data a in which the mixing ratios of N cell types are represented by an N-dimensional vector is obtained.

As described above, the mixing ratio prediction device 10 according to the embodiment of the present invention can obtain the mixing ratio prediction data a from the bulk cell expression level data y. As described above, unlike the experiment using cell counter in the related art, the mixing ratio prediction device 10 according to the embodiment of the present invention can directly predict the mixing ratio of each cell type contained in the bulk cell from the gene expression levels in the bulk cell.

### Example of Comparison with Method in the Related Art

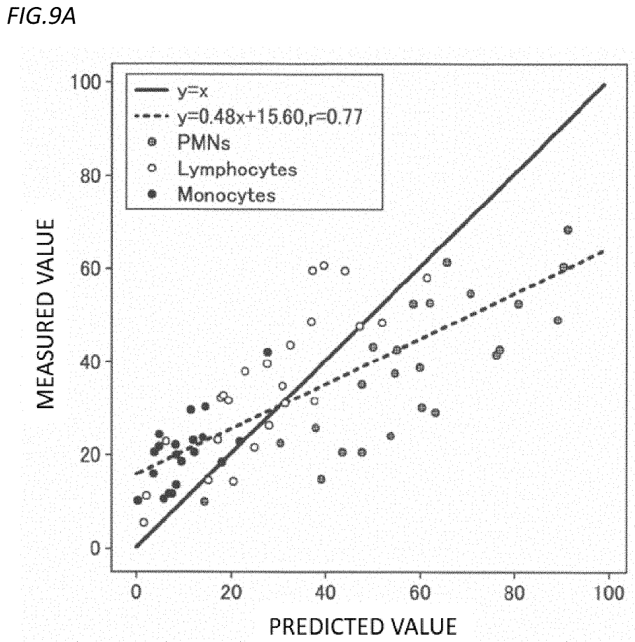

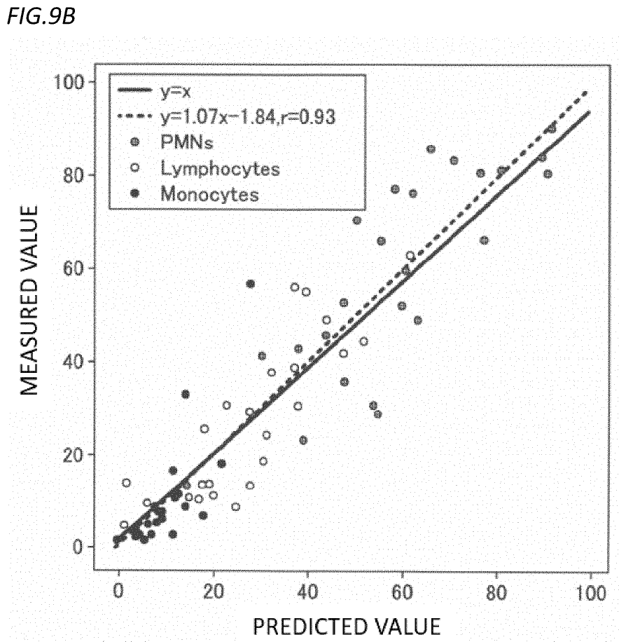

A description will be given below of a comparison example of prediction accuracy between a method in the related art and the method according to the embodiment of the present invention with reference to FIG. 9A and 9B. FIG. 9A and 9B are diagrams showing an example of comparison with the method in the related art. In the example shown in FIG. 9A and 9B, the GSE20300 dataset was used as the bulk cell expression level data y.

FIG. 9A is a diagram where a relationship between measured and predicted values of a mixing ratio when CIBERSORT described in Non Patent Literature 1 described above is used as the method in the related art is plotted as a point. On the other hand, FIG. 9B is a diagram where a relationship between measured and predicted values of a mixing ratio when the method according to the embodiment of the present invention is used is plotted as a point. Note that, in FIGS. 9A and 9B, in order to facilitate comparison, 19 cell types out of 22 cell types were collectively referred to as “PMNs”, and these “PMNs”, a cell type “Lymphocytes”, and a cell type “monocytes” were plotted. Further, a cell type “Eosinophils”, one of 22 cell types, was excluded.

In the example shown in FIG. 9A, the regression line obtained from each plotted point is represented by y=0.48x+15.60, and the correlation coefficient is r=0.77. On the other hand, in the example shown in FIG. 9B, the regression line obtained from each point is represented by y=1.07x−1.84, and the correlation coefficient is r=0.93. Note that the closer the regression line is to y=x, the higher the prediction accuracy.

This shows that the mixing ratio prediction device 10 according to the embodiment of the present invention can predict the mixing ratio with high accuracy compared to the method in the related art such as CIBERSORT.

### SUMMARY

As described above, the mixing ratio prediction device 10 according to the embodiment of the present invention is capable of predicting, with the predictor implemented by the learned neural network, the mixing ratio of each cell type contained in the bulk cell from data indicating the gene expression levels in the bulk cell. In order to cause this predictor to learn, the mixing ratio prediction device 10 according to the embodiment of the present invention generates, from data indicating the gene expression levels of each cell type, the learning data which is a set of data indicating the gene expression levels in the virtual bulk cell and data indicating the mixing ratio of each cell type contained in the virtual bulk cell.

Therefore, the mixing ratio prediction device 10 according to the embodiment of the present invention is capable of easily creating the learning dataset even when it is difficult to measure the gene expression levels in the bulk cell and the mixing ratio of each cell type contained in the bulk cell by experiment or the like.

Further, the mixing ratio prediction device 10 according to the embodiment of the present invention is capable of predicting the mixing ratio with high accuracy by using the predictor learned as described above even when, for example, the gene expression level cannot be estimated to have linearity. Herein, a case where the gene expression level can be estimated to have linearity corresponds to a case where the gene expression level in the bulk cell can be expressed by the sum of the products of the gene expression level in each cell type and the mixing ratio of the cell type (further including a case where the gene expression level in the bulk cell can be expressed by the sum of the above-described sum and the term representing noise).

Note that, according to the embodiment of the present invention, the case of predicting the mixing ratio of each cell type contained in the bulk cell has been described, but the present invention is applicable to not only such a case, but also a case of, for example, predicting the mixing ratio of each component contained in an unknown chemical substance. Further, the embodiment of the present invention is applicable to any task of estimating the mixing ratio of each unknown signal in an issue setting where a signal representing a pure object (or element) can be obtained.

Further, according to the above-described embodiment, the dataset creation module 101 is provided in the mixing ratio prediction device 10, but the present invention is not limited to such a configuration. That is, the dataset creation module 101, the learning module 102, and the prediction module 103 may be provided separately as a dataset creation device, a learning device, and a prediction device, respectively.

The present invention is not limited to the embodiment disclosed in detail above, and various modifications or changes can be made without departing from the scope of the claims.

### EXPLANATIONS OF REFERENCE NUMBERS

10 mixing ratio prediction device

101 dataset creation module

102 learning module

103 prediction module

111 mixing ratio generator

112 bulk cell creator

113 learning data creator

What is claimed is:

1. A learning method for predicting mixing ratios of elements, performed at a computing system including one or more computing devices, each computing device having one or more processors and memory, the learning method comprising:
receiving a set of data for a predetermined plurality of elements, the data including, for each of the elements, a respective set of expression levels for each of a predetermined plurality of components that are included in the respective element; and
using the set of data, training a machine learning model to predict a proportion of at least one element in a bulk sample of the plurality of elements in response to input of a respective expression level for each of the plurality of components included in elements of the bulk sample.

2. The learning method of claim 1, wherein training the machine learning model uses a plurality of virtual training vectors, each of the virtual training vectors generated according to (i) a respective distinct virtual mixing ratio that specifies non-zero proportions for two or more of the predetermined elements and (ii) the expression level for each component of the elements with non-zero proportions.

3. The learning method of claim 2, wherein the set of data comprises a first element and a second element, and each of the virtual mixing ratios includes a non-zero proportion for the first element and for the second element.

4. The learning method of claim 2, wherein the set of data comprises a first element, a second element, and a third element, and each of the virtual mixing ratios includes a non-zero proportion only for the first element and for the second element.

5. The learning method of claim 2, wherein one or more of the virtual mixing ratios is a value determined based on a random number.

6. The learning method of claim 2, wherein each virtual training vector includes a virtual expression level for one or more components, calculated as a linear combination of the expression levels for the respective component in each of the elements according to the respective proportions specified by the respective mixing ratio.

7. The learning method of claim 6, wherein each virtual expression level is a value obtained by normalizing a value that results from multiplying the respective virtual mixing ratio by predetermined noise and the expression level in each of the elements.

8. The learning method of claim 1, wherein the elements are cell types.

9. The learning method of claim 8, wherein each expression level is a respective gene expression level.

10. The learning method of claim 1, wherein the elements are chemical substances.

11. The learning method of claim 1, wherein the machine learning model is a neural network.

12. A prediction method for predicting mixing ratios of elements, performed at a computing system including one or more computing devices, each computing device having one or more processors and memory, the prediction method comprising:
predicting a proportion of at least one element in a group of elements, each element having a respective set of components, the prediction applying a trained machine learning model to supplied group expression level data indicating a respective aggregate expression level for each component present in at least one of the elements in the group of elements.

13. The prediction method of claim 12, wherein the elements are cell types.

14. The prediction method of claim 12, wherein each expression level is a respective gene expression level.

15. The prediction method of claim 12, wherein the elements are chemical substances.

16. The prediction method of claim 12, further comprising predicting a proportion of each element contained in the group.

17. The prediction method of claim 16, wherein the elements are chemical substances.

18. A prediction device for predicting mixing ratios of elements, comprising:
memory;
one or more processors; and
one or more programs stored in the memory, the one or more programs including instructions for:
predicting a proportion of at least one element in a group of elements, each element having a respective set of components, the prediction applying a trained machine learning model to supplied group expression level data indicating a respective aggregate expression level for each component present in at least one of the elements in the group of elements.

19. The prediction device of claim 18, wherein the elements are cell types and each expression level is a respective gene expression level.

20. The prediction device of claim 18, wherein the machine learning model is a neural network.
